(19) United States
(12) Patent Application Publication
(10) Pub. No.: US 2022/0133833 A1
(43) Pub. Date: May 5, 2022

(54) USE OF GRAPE AND/OR SOY SUPPLEMENTS AGAINST COVID-19 AND 92 OTHER VIRUSES AND BACTERIA INCLUDING OTHER UNKNOWN VIRUSES AND BACTERIA

(71) Applicant: Kieu Hoang, Westlake Village, CA (US)

(72) Inventor: Kieu Hoang, Westlake Village, CA (US)

(21) Appl. No.: 17/370,994

(22) Filed: Jul. 8, 2021

Related U.S. Application Data
(60) Provisional application No. 63/049,224, filed on Jul. 8, 2020.

(51) Int. Cl. A61K 36/899; A61K 36/87; A61K 36/48; A61P 31/16; A61P 31/14; A23L 33/00; A23L 33/105

(52) U.S. Cl. CPC A61K 36/899; A61K 36/87; A61K 36/48; A23V 2002/00; A61P 31/14; A23L 33/40; A23L 33/105; A61P 31/16

(57) ABSTRACT

The invention relates to RAAS Nutritionals food functional dietary supplements derived from soybean (Glycine max), grape (vitis vinifera) and rice (Oryza sativa) for prophylaxis and therapeutic treatment of virus SARS-CoV-2, Acinetobacter baumanii, Bordetella pertussis, parapertusis, bronchiseptica, Chlamydia trachomatis, Coronavirus-299E, Coronavirus NL63, Coronavirus OC43, Coronavirus HKU1, Cytomegalovirus CMV, Cytomegalovirus Human Herpesvirus-5, Enterobacter aerogenes, Enterobacter cloacae, Enterovirus D, Fusobacterium nucleatum, Fusobacterium necrophorum, Herpes simplex virus 1 (HSV-1), Herpes simplex virus 2 (HSV-2), Human metapneumovirus, Klebisella pneumoniae, Klebisella oxytoca, Moraxella catarrhalis, Meisseria gonorrhoeae, Proteus mirabilis, Proteus vulgaris, Respiratory syncytial virus, Serratia marcescens, Staphylococcus aureus, Streptococcus pneumoniae, Treponema pallidum, CTX-M1 (15), CTX-M2 (2), CTX-M9 (9), CTX-M8/25 groups, ermB, ermC, mefA, mecA, Per-lNEB-1/GES-1 Groups, SHV, KPC Groups, VanA, VanB, Adenovirus Respiratory HAdV-B, Candida Albicans, Candida glabrata, Candida parapsilosis, Candida tropicalis, Chlamydophila pneumoniae, EBV(Epstein Barr Virus/Human Herpes Virus 4), Enterovirus A, Enterovirus B, Enterovirus C, Escherichia coli, Haemophilus influenzae, Human herpesvirus 3 (varicella zoster virus), Influenza A, Inluenza A/H1, Influenza A/H3, Influenza A/H1-2009, Influenza B, Legionella pneumophila, Mycoplasma pneumoniae, Parainfluenza virus 1, Parainfluenza 2 virus, Parainfluenza 3 virus, Parainfluenza 4 virus, Pseudomonas aeruginosa, Rhinovirus A, Rhinovirus B, Rhinovirus C, Staphylococcus coagulase negative epidermis, Staphylococcus coagulase negative haemolyticus, Staphylococcus coagulase negative lugdunensis, Staphylococcus agalactiae (Group B Strep), Staphylococcus pyogenes (Group A strep), ACT, MIR, FOX, ACC, dfr A1, dfr A5, sul 1 probe, sul 2 probe, IMP, NDM, VIM Groups, OXA-48, OXA-51, qnrA1, qnrA2, qnrB2, tet B and tet M viruses, bacteria and unknown new viruses and bacterias that we classify as Not Otherwise Specified (NOS).

### PRIORITY CLAIM

This application claims the benefit of U.S. Provisional Application Ser. No. 63/049,224, filed Jul. 8, 2020, which application is incorporated herein by reference.

The invention relates to RAAS Nutritionals food functional dietary supplements derived from soybean (Glycine max), grape (vitis vinifera) and rice (Oryza sativa) for prophylaxis and therapeutic treatment of virus SARS-CoV-2, Acinetobacter baumanii, Bordetella pertussis, parapertusis, bronchiseptica, Chlamydia trachomatis, Coronavirus-299E, Coronavirus NL63, Coronavirus OC43, Coronavirus HKU1, Cytomegalovirus CMV, Cytomegalovirus Human Herpesvirus-5, Enterobacter aerogenes, Enterobacter cloacae, Enterovirus D, Fusobacterium nucleatum, Fusobacterium necrophorum, Herpes simplex virus 1 (HSV-1), Herpes simplex virus 2 (HSV-2), Human metapneumovirus, Klebisella pneumoniae, Klebisella oxytoca, Moraxella catarrhalis, Meisseria gonorrhoeae, Proteus mirabilis, Proteus vulgaris, Respiratory syncytial virus, Serratia marcescens, Staphylococcus aureus, Streptococcus pneumoniae, Treponema pallidum, CTX-M1 (15), CTX-M2 (2), CTX-M9 (9), CTX-M8/25 groups, ermB, ermC, mefA, mecA, Per-1NEB-1/GES-1 Groups, SHV, KPC Groups, VanA, VanB, Adenovirus Respiratory HAdV-B, Candida Albicans, Candida glabrata, Candida parapsilosis, Candida tropicalis, Chlamydophila pneumoniae, EBV(Epstein Barr Virus/Human Herpes Virus 4), Enterovirus A, Enterovirus B, Enterovirus C, Escherichia coli, Haemophilus influenzae, Human herpesvirus 3 (varicella zoster virus), Influenza A, Inluenza A/H1, Influenza A/H3, Influenza A/H1-2009, Influenza B, Legionella pneumophila, Mycoplasma pneumoniae, Parainfluenza virus 1, Parainfluenza 2 virus, Parainfluenza 3 virus, Parainfluenza 4 virus, Pseudomonas aeruginosa, Rhinovirus A, Rhinovirus B, Rhinovirus C, Staphylococcus coagulase negative epidermis, Staphylococcus coagulase negative haemolyticus, Staphylococcus coagulase negative lugdunensis, Staphylococcus agalactiae (Group B Strep), Staphylococcus pyogenes (Group A strep), ACT, MIR, FOX, ACC, dfr A1, dfr A5, sul 1 probe, sul 2 probe, IMP, NDM, VIM Groups, OXA-48, OXA-51, qnrA1, qnrA2, qnrB2, tet B and tet M viruses, bacteria and unknown new viruses and bacterias that we classify as Not Otherwise Specified (NOS).

### BACKGROUND OF THE INVENTION

During the COVID-19 pandemic from December 2019 in China, the author has launched a medical observation for 2,500 patients, mainly the Vietnamese American, who have returned to Vietnam for new years celebrations, do any of them got COVID-19 virus? About 50 of them may have had the viruses and bacterias as listed in the abstract, but none of them died and all recovered.

In addition, the author conducted a medical observation at first for 17 kinds of different viruses and 4 kinds of bacterias including Adenovirus, Coronavirus HKU1, Coronavirus NL63, Coronavirus 229E, Coronavirus OC43, Human Metapneumovirus, Human Rhinovirus/Enterovirus, Influenza A, Influenza A/H1, Influenza A/H3, Influenza A/H1-2009, Influenza B, Parainfluenza Virus 1, Parainfluenza Virus 2, Parainfluenza Virus 3, Parainfluenza Virus 4, Respiratory Syncytial Virus,Bordetella parapertussis, Bordetella pertussis, Chlamydia pneumoniaeand Mycoplasma pneumoniae.

### SUMMARY OF THE INVENTION

RAAS Nutritionals food functional dietary supplement, made from concentrated natural botanical ingredients derived from soybean (Glycine max), grape (vitis vinifera) and rice (Oryza sativa) for prophylaxis and therapeutic treatment of virus SARS-CoV-2, Acinetobacter baumanii, Bordetella pertussis, parapertusis, bronchiseptica, Chlamydia trachomatis, Coronavirus-299E, Coronavirus NL63, Coronavirus OC43, Coronavirus HKU1, Cytomegalovirus CMV, Cytomegalovirus Human Herpesvirus-5, Enterobacter aerogenes, Enterobacter cloacae, Enterovirus D, Fusobacterium nucleatum, Fusobacterium necrophorum, Herpes simplex virus 1 (HSV-1), Herpes simplex virus 2 (HSV-2), Human metapneumovirus, Klebisella pneumoniae, Klebisella oxytoca, Moraxella catarrhalis, Meisseria gonorrhoeae, Proteus mirabilis, Proteus vulgaris, Respiratory syncytial virus, Serratia marcescens, Staphylococcus aureus, Streptococcus pneumoniae, Treponema pallidum, CTX-M1 (15), CTX-M2 (2), CTX-M9 (9), CTX-M8/25 groups, ermB, ermC, mefA, mecA, Per-UVEB-1/GES-1 Groups, SHV, KPC Groups, VanA, VanB, Adenovirus Respiratory HAdV-B, Candida Albicans, Candida glabrata, Candida parapsilosis, Candida tropicalis, Chlamydophila pneumoniae, EBV(Epstein Barr Virus/Human Herpes Virus 4), Enterovirus A, Enterovirus B, Enterovirus C, Escherichia coli, Haemophilus influenzae, Human herpesvirus 3 (varicella zoster virus), Influenza A, Inluenza A/H1, Influenza A/H3, Influenza A/H1-2009, Influenza B, Legionella pneumophila, Mycoplasma pneumoniae, Parainfluenza virus 1, Parainfluenza 2 virus, Parainfluenza 3 virus, Parainfluenza 4 virus, Pseudomonas aeruginosa, Rhinovirus A, Rhinovirus B, Rhinovirus C, Staphylococcus coagulase negative epidermis, Staphylococcus coagulase negative haemolyticus, Staphylococcus coagulase negative lugdunensis, Staphylococcus agalactiae (Group B Strep), Staphylococcus pyogenes (Group A strep), ACT, MIR, FOX, ACC, dfr A1, dfr A5, sul 1 probe, sul 2 probe, IMP, NDM, VIM Groups, OXA-48, OXA-51, qnrA1, qnrA2, qnrB2, tet B and tet M viruses and bacteria.

### BRIEF DESCRIPTION OF THE DRAWINGS:

FIG. 1—Pictures of plant cells, animal cells and human cells.

FIG. 2—Chart of cell count in RAAS Nutritionals products.

FIG. 3—Polyacrylamide gel electrophoresis (SDS-PAGE) of human albumin, human immunoglobulin, human APOA-1, grape juice KHJ.

FIG. 4—Polyacrylamide gel electrophoresis (SDS-PAGE) of human albumin, human immunoglobulin, human APOA-1 & supernatant edamame.

FIG. 5—Polyacrylamide gel electrophoresis (SDS-PAGE) of human albumin, human immunoglobulin, human APOA-1 & paste edamame.

FIG. 6—Polyacrylamide gel electrophoresis (SDS-PAGE) of human albumin, human immunoglobulin, human APOA-1 & soybean supernatant and paste.

FIG. 7—Polyacrylamide gel electrophoresis (SDS-PAGE) of human albumin, human immunoglobulin, human APOA-1 & soybean concentrate & permeate.

FIG. 8—Serum protein electrophoresis of human albumin, human immunoglobulin, human APOA-1 & Kunakin® (soybean)

FIG. 9—Protein molecular weight analysis for brown, white & sticky rice.

FIG. 10—Summary of in vitro study for 14 RAAS Nutritionals products against HBV, HCV, H1N1 & HIV.

FIG. 11—In vivo survival rate of RAAS therapeutic product AFOD in H1N1 infected mice.

FIG. 12—Summary chart of 13 positive COVID-19 patients

FIG. 13—Viral load reduction recovery rate

FIG. 14-15—Age group of 13 COVID-19 positive patients

FIG. 16 Prophylaxis and 13 therapeutic groups

FIG. 17—Summary of positive COVID-19 patients with bacteria.

FIG. 18—NOS in COVID-19 positive group

FIG. 19—Summary 9 NOS patients in 26 negative COVID-19 group

### DETAILED DESCRIPTION OF THE INVENTION

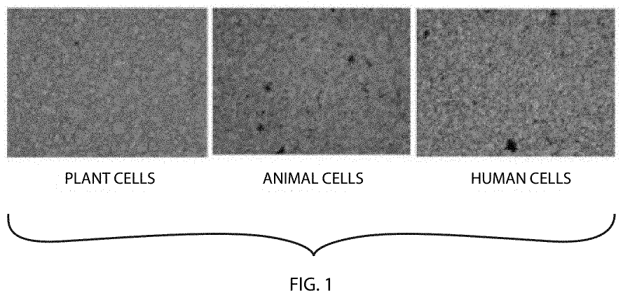

Plant cells, Animal cells and Human share the same structure. The cells found in RAAS Nutritionals food functional dietary supplements are named “KH Good Healthy” cells which synthesize “KH Good Healthy” proteins. The cells never die. Fat, glucose equals protein. Protein equals cell. What we learned from text books and FDA conclude that when the protein goes through filtration step which removes the cover of the cell and leaves the protein like human albumin or human immunoglobulin, which are CELLS equals PROTEIN. (FIG. 1)

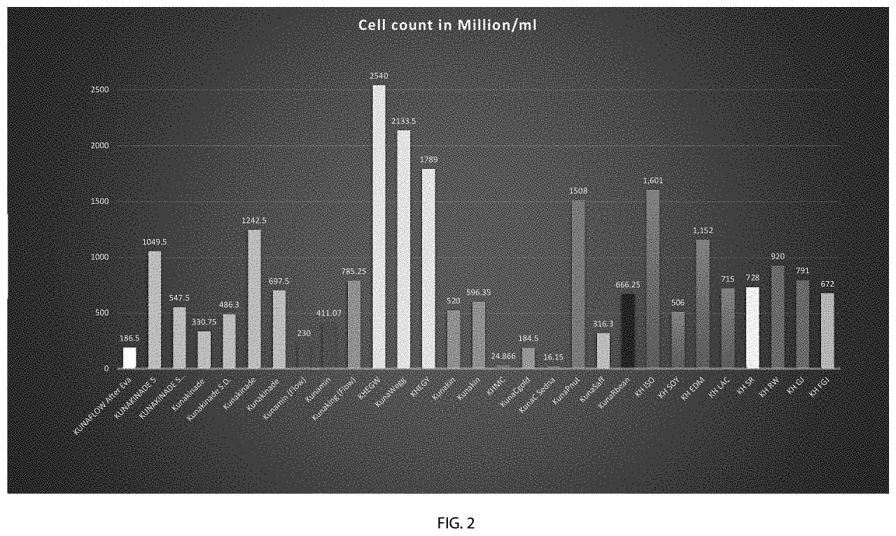

In all our of our products, each contains a minimum number of cells from 16.5 million cells/mL up to the maximum 2.5 billion cells/mL. (FIG. 2)

These complex compounds of natural “KH Good Healthy” proteins which contain good healthy cells which are absorbed into the blood stream to send signal to the body to produce new cells that are healthy. It also sends the RNA signal to the DAMAGED, SICK, AND BAD CELLS that triggers that synthesis of good proteins that transform these cells to become GOOD healthy cells. “KH Good Healthy” proteins also send the RNA signal to the other currently undamaged cells to synthesize good proteins to protect them from being DAMAGED, INFECTED and PRONE to DNA and other cellular alterations.

RAAS Nutritionals food functional dietary supplements derived from soy, grapes and rice that contain high concentrations of Apo-A1, Immunoglobulin and Albumin like proteins. Unlike traditional human plasma derived therapies that limit the product to one single protein, RAAS Nutritionals supplements contain up to 5,055 proteins.

| | New | Discovered | Total |
|---|---|---|---|
| Botanical name | Protein | Protein | Proteins |
| Grape (vitis vinifera) | 959 | 340 | 1,299 |
| Soybean (Glycine max) | 125 | 233 | 358 |
| Younger Soy Edamame (Glycine max) | 843 | 380 | 1,223 |
| Rice (Oryza sativa) | 569 | 1,606 | 2,175 |

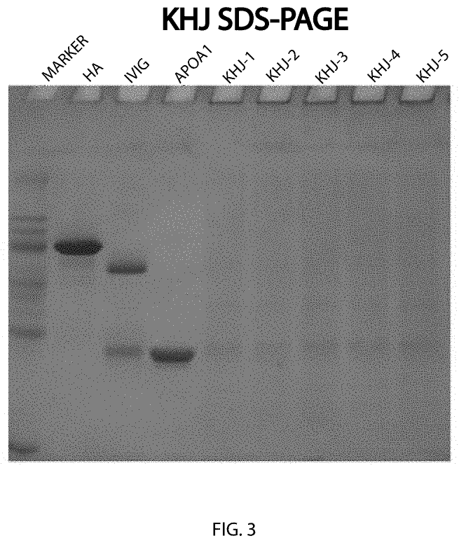

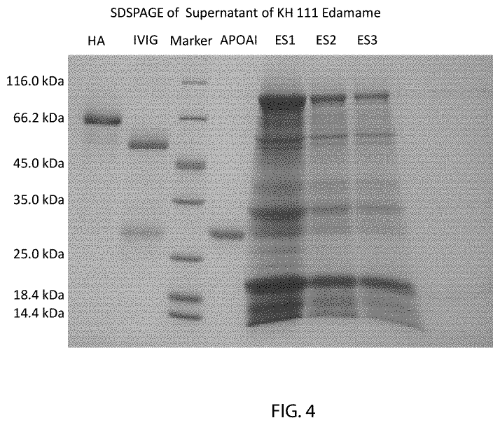

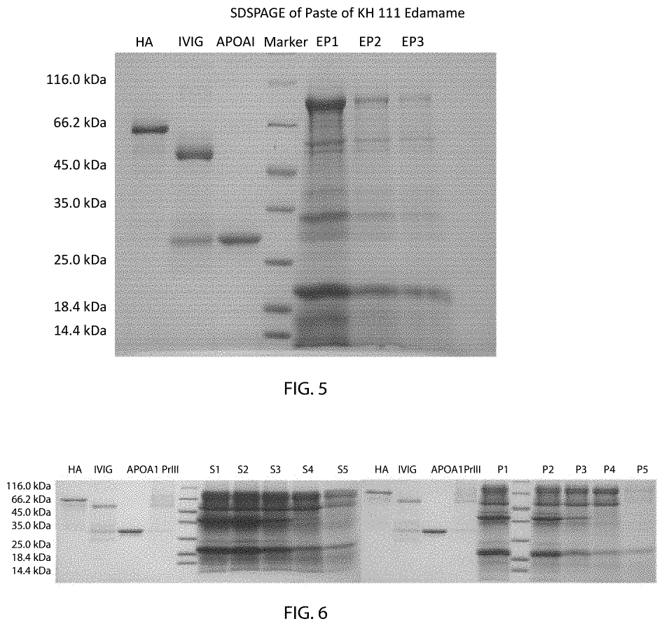

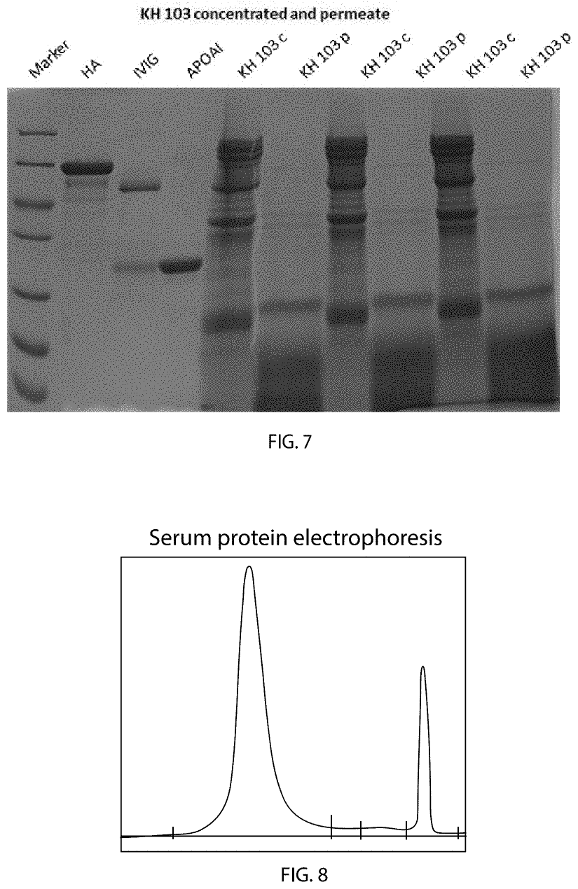

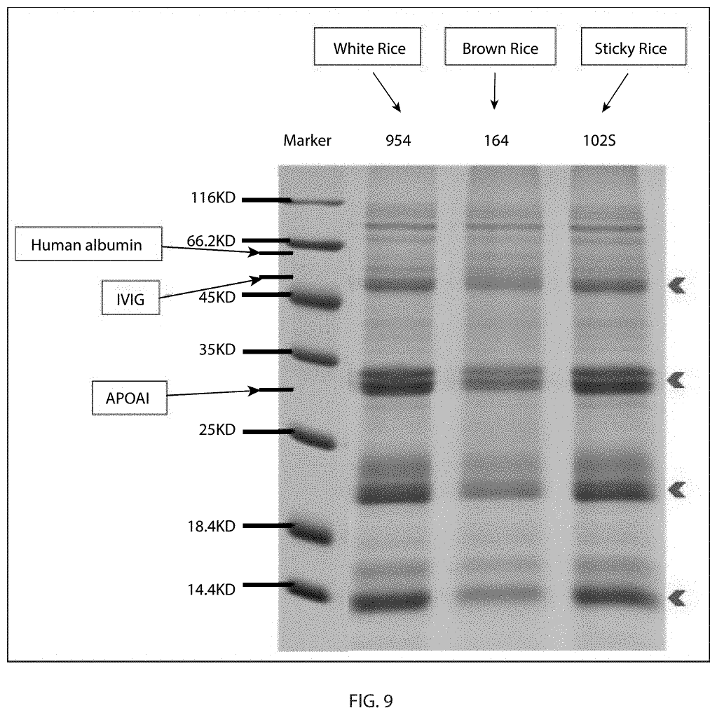

Grape, soybeans, edamame and rice contain proteins like APOA-1, immunoglobulin and albumin. APOA-1 is the HDL, which is a very important protein for a human being. The next important proteins are immunoglobulin and albumin. (FIGS. 3, 4, 5, 6, 7, 8 & 9)

### In Vitro Studies

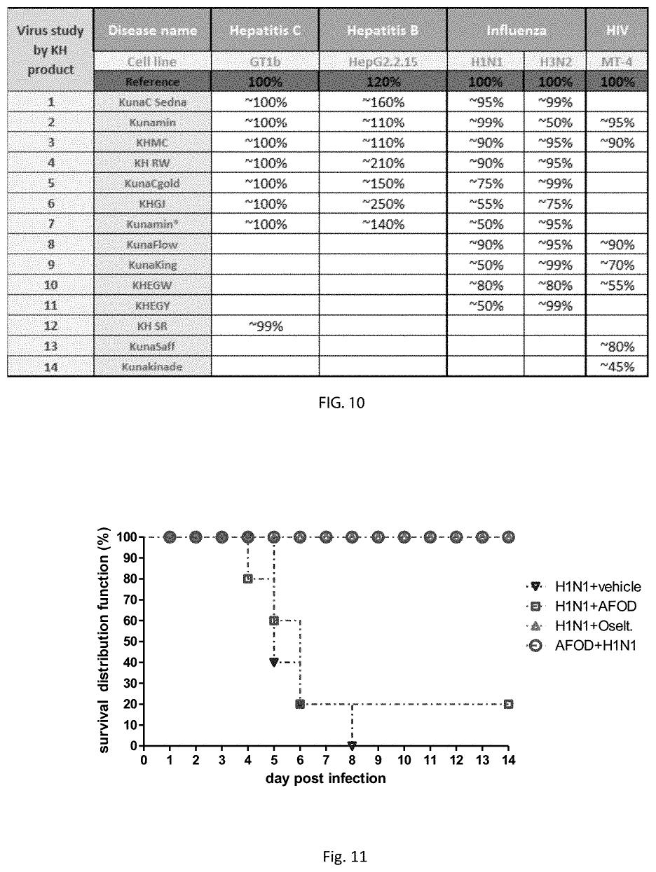

RAAS Nutritionals has 14 different products tested against HCV, HBV, H1N1, H3N2 and HIV. Each of those can be found to be effectively used for therapeutic and prophylaxis. (FIG. 10)

### In Vivo Studies

We have found that APOA-1 from human plasma can prevent and treat H1N1 virus. In our study performed at Wuxi Pharma, one of the top ten CRO's in the world, we found that RAAS-2 (APOA-1) can prevent 100% of the Bird Flu Epidemiology in Mice.

Impressively one-week preventive treatment with 0.2 ml/0.4 ml/mouse iv/ip QD of RAAS-2 (APOA-1) totally protected H1N1-challenged mice from death and body weight loss till the end of this study. The protection of body weight loss by the preventive treatment of RAAS-2 (APOA-1) is even better than that by oseltamivir treatment. However, the therapeutic treatment with 0.2 ml/0.4 ml iv/ip QD of RAAS-2 only protected one mouse out of 5 mice in the group from death and partial body weight loss of all 5 mice days 2 to 5 post H1N1 infection. Other 4 mice in this group died days 4 to 6 post H1N1 infection. In addition, some of the mice in the RAAS-2 therapeutic group (G2) had haematuria day 5 post H1N1 challenge and afterward, indicating that the dose used in this group was beyond mouse tolerance in H1N1 challenge status. (FIG. 11)

RAAS Nutritionals food functional dietary supplements are approved for sale over the counter by the FDA and have been on the market since 2013. Therefore, there is no need for clinical trials. FDA Registration No.: 11982611580

RAAS has conducted a cold and viral flu medical observation on 2,500 patients who are also participating in the different diseases and cancers medical observation. The author found that none of the 2,500 patients who had the cold/flu died because of it eventhough the patients all suffered from underlying medical conditions like diabtes, chronic kidney disease, Hepatits B, Hepatitis C and Hypercholesterolemia (atherosclerosis). 98% of these 2,500 patients are Vietnamese American who travel back and forth between United States and Vietnam during the Vietnamese new year period, the author questions himself if any of them had COVID-19?

### Medical Observation

In addition to the large medical observation for 2,500 patients, the author together the Medical Director of RAAS Nutritionals, LLC, who has an Urgent Care practice which is allowed to test for COVID-19 once the FDA approved the COVID-19 testing from private laboratories. The author was so grateful to the FDA which allowed 2,000 laboratories to begin testing for COVID-19. This gave the opportunity at RAAS testing site to test for COVID-19.

RAAS Nutritionals medical observation enrolled 77 patients with symptoms of cold, flu, shortness of breath, fever, sore throat, sneezing, inflammation and others. Most COVID-19 patient's symptoms will be the loss of appetite. The most affected are the eyes according to ophthalmologists like Dr. Li Wenliang, who became the whistleblower for the COVID-19 outbreak. In two studies, one in China with 214 COVID-19 patients, one third of the patients experienced brain injuries like loss of taste and smell, dizziness, headaches, unconsciousness and strokes. In another study at Mt. Sinai hospital, they had 45 strokes in 4 weeks which is nearly three times more than the normal average. Half of the patients were COVID-19 positive. The average age of the stroke patients was 12 years younger than typical victims.

During the study period from patient #1 to patient #28 there was no COVID-19 testing available, so we had to use BioFire Film Array testing panel for Adenovirus, Coronavirus HKU 1, Coronavirus NL63, Coronavirus 229E, Coronavirus OC43, Human Metapneumovirus, Human Rhinovirus/Enterovirus, Influenza A, Influenza A/H1, Influenza A/H3, Influenza A/H1-2009, Influenza B, Parainfluenza Virus 1, Parainfluenza Virus 2, Parainfluenza Virus 3, Parainfluenza Virus 4, Respiratory Syncytial Virus, Bordetella parapertussis, Bordetella pertussis, Chlamydia pneumoniae and Mycoplasma pneumoniae. We had detected six patients with NOS (Not Otherwise Specified) viruses or bacteria. One of these NOS patients, patient #28, was later diagnosed positive with COVID-19. From patient #28 to #77, thirteen of them have been infected with COVID-19.

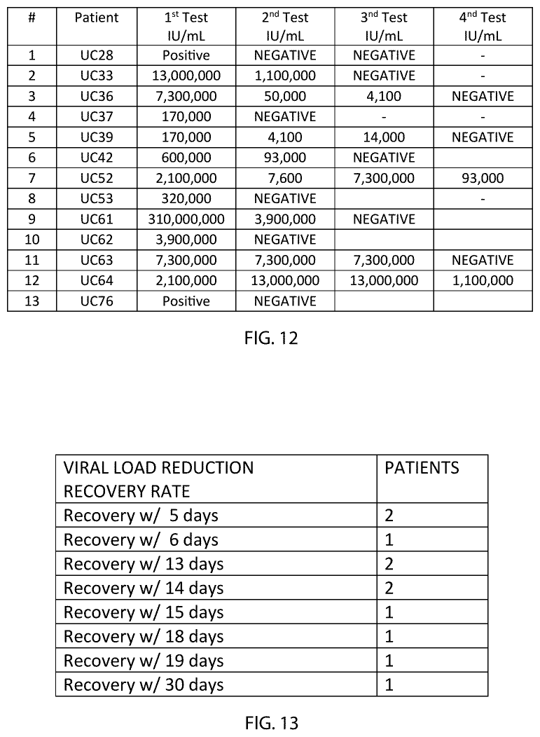

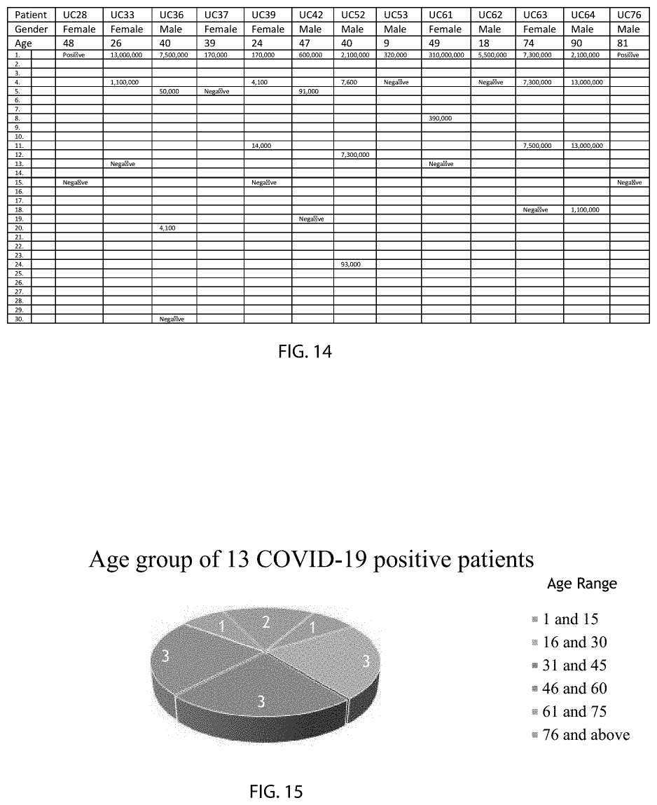

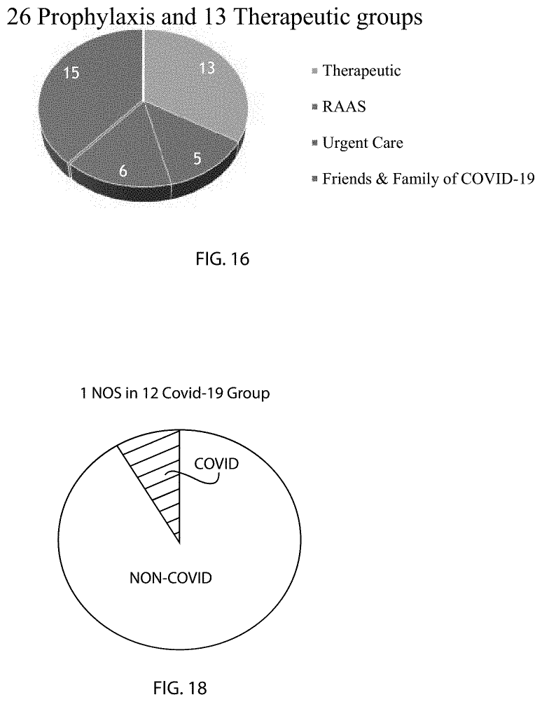

Patient numbers for positive COVID-19 are #28, #33, #36, #37, #39, #42, #52, #53, #61, #62, #63, #64 and #76. Patient #61 with the highest viral load of 310,000,000 which was reduced to 3,900,000 copies/mL within seven days. Eleven of these patients have been confirmed negative after treatment. Two patients are still under treatment. (FIG. 12)

*Patient UC63 & patient UC64 are within the oldest in the group. One female 74 y/o and male 90 y/o. They did not take the food functional dietary supplements because the doctor prescribed antibiotics which caused the stomach to become upset therefore cannot take the supplements.

Viral Load Reduction Recovery Rate

Among the 13 positive COVID-19 patients, there were 7 male and 6 female patients. The age of the patients ranges from the youngest 9 years old to the oldest 90 years old. 2 patients (UC53, UC62) recovered within 5 days, 1 patient (UC37) recovered within 6 days, 2 patients (UC33, UC61) recovered within 13 days, 2 patients (UC28, UC39) recovered within 14 days, 1 patient (UC76) recovered within 15 days, 1 patient (UC63) recovered within 18 days, 1 patient (UC42) recovered within 19 days and 1 patient (UC36) recovered within 30 days. (FIG. 13, 14, 15, 16)

Twenty-six of the patients have tested negative for COVID-19, all these patients are either family members or contacts of those infected as well as eight employees of the Westlake Urgent Care including the doctor in charge. Five of these are the family members of COVID-19 patient who is in intensive care unit at the Simi Valley Seventh Adventist Hospital suffering serious pneumonia.

The COVID-19 patients can also have bacterial infections. In our medical observation of 13 cases of COVID-19, we have found 5 cases with the following: #52 Fusobacterium nucleatum, necrophorum, streptococcus pneumoniae, #63 Haemophilus influenzae, #53 streptococcus pyogenes, mycoplasma pneumoniae, Streptococcus pneumoniae, #39 Staphylococcus aureus, #36 Moraxella catarrhalis. One COVID-19 negative patient UC51 had Staphylococcus (coagulase negative: epidermidis, haemolyticus, lugdunesis, saprophyticus). In a very small study of 12 COVID-19 positive patients, 41% had a combination of COVID-19 and bacteria. In the study we observed 12 patients out of 77 with NOS mysterious virus or bacteria or 16.5%.

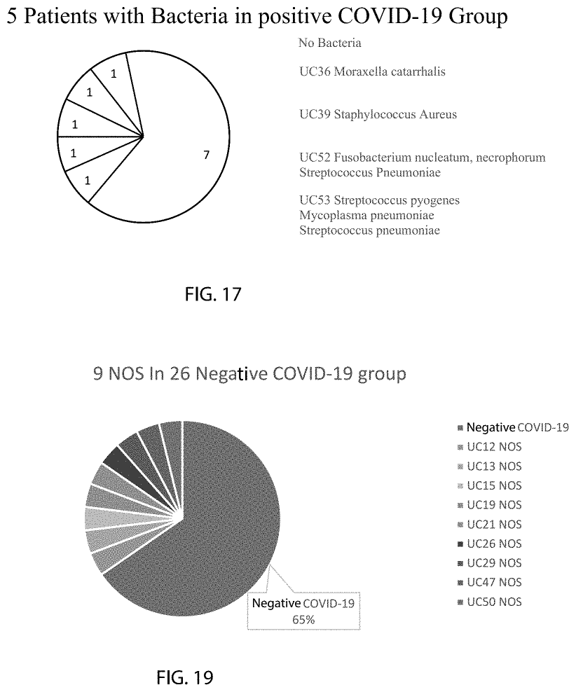

COVID-19 virus patients have been infected by bacteria. Five out of twelve patients (42%) have both COVID-19 and the following types of bacteria: Moraxella catarrhalis, Staphylococcus Aureus, Fusobacterium nucleatum, necrophorum, Streptococcus Pneumoniae, Streptococcus pyogenes, Mycoplasma pneumoniae and Haemophilus Influenzae. (FIG. 17)

NOS (Non-Otherwise Specified) virus or bacteria that cannot be identified from 93 viruses and bacteria including COVID-19.

COVID-19 has mutated into at least 32 different strains and that is why in our medical observation we have found at least one NOS in 13 positive COVID-19 group. (FIG. 18)

9 NOS in 26 negative COVID-19 group. The 26 negative COVID-19 group are the relatives and friends of the positive COVID-19 patients. The group of negative for COVID-19 without NOS is mostly from the prophylaxis group including the Doctor and staff of the Westlake Village Urgent Care. The other group is the RAAS group.

Six of the NOS were detected when we used the Biofire FilmArray Testing panel which includes 17 viruses and 4 bacteria. Adenovirus, Coronavirus HKU1, Coronavirus NL63, Coronavirus 229E, Coronavirus OC43, Human Metapneumovirus, Human Rhinovirus/Enterovirus, Influenza A, Influenza A/H1, Influenza A/H3, Influenza A/H1-2009, Influenza B, Parainfluenza Virus 1, Parainfluenza Virus 2, Parainfluenza Virus 3, Parainfluenza Virus 4, Respiratory Syncytial Virus, Bordetella parapertussis, Bordetella pertussis, Chlamydia pneumoniae and Mycoplasma pneumoniae. None of these 17 viruses and 4 bacteria's were detected. Therefore, we named the virus or bacteria NOS (Non-Otherwise Specified).

There are several more with NOS from patients 28 to 74, actually we do not know what NOS is from our medical observation involving a lot of different people like Vietnamese, Chinese, Korean, Caucasian and Hispanic. Unfortunately, we did not have any African American patients, who suffer the most in the COVID-19 pandemic. (FIG. 19)

UC29 first test was NOS then returned for second test which confirmed negative COVID-19 after taking RAAS Nutritionals food functional dietary supplements.

Patient UC47, one of the RAAS employees experiencing flu like symptoms tested NOS after he had been taking RAAS Nutritionals food functional dietary supplements for prophylaxis.

During COVID-19 pandemic 50% of RAAS employees participating in this medical observation and occupy 13% of the total number of patients including Kieu Hoang's two sons had Influenza A, Influenza B, H. Rhinovirus, Enterovirus, RSV and NOS.

Prior to this study, we have done a medical observation for 2,500 patients at four different locations and found that out of 623 completed patients, 402 of them had no cold or flu. 47 of them had cold or flu. 174 patients we were not able to confirm whether they had cold or flu. 2,500 mainly Vietnamese American who travel back and forth Vietnam using all the Chinese, Korean, Japanese and Hong Kong airlines, the author wonders if anyone got COVID-19?

Individual studies for hepatitis B, hepatitis C, CKD, lipid panel, pre diabetes, diabetes and severe diabetes have been completed separately.

I claim:

1. A method of prophylaxis and therapeutic treatment of SARS-CoV-2, Acinetobacter baumanii, Bordetella pertussis, parapertusis, bronchiseptica, Chlamydia trachomatis, Coronavirus-299E, Coronavirus NL63, Coronavirus OC43, Coronavirus HKU1, Cytomegalovirus CMV, Cytomegalovirus Human Herpesvirus-5, Enterobacter aerogenes, Enterobacter cloacae, Enterovirus D, Fusobacterium nucleatum, Fusobacterium necrophorum, Herpes simplex virus 1 (HSV-1), Herpes simplex virus 2 (HSV-2), Human metapneumovirus, Klebisella pneumoniae, Klebisella oxytoca, Moraxella catarrhalis, Meisseria gonorrhoeae, Proteus mirabilis, Proteus vulgaris, Respiratory syncytial virus, Serratia marcescens, Staphylococcus aureus, Streptococcus pneumoniae, Treponema pallidum, CTX-M1 (15), CTX-M2 (2), CTX-M9 (9), CTX-M8/25 groups, ermB, ermC, mefA, mecA, Per-UVEB-1/GES-1 Groups, SHV, KPC Groups, VanA, VanB, Adenovirus Respiratory HAdV-B, Candida Albicans, Candida glabrata, Candida parapsilosis, Candida tropicalis, Chlamydophila pneumoniae, EBV(Epstein Barr Virus/Human Herpes Virus 4), Enterovirus A, Enterovirus B, Enterovirus C, Escherichia coli, Haemophilus influenzae, Human herpesvirus 3 (varicella zoster virus), Influenza A, Inluenza A/H1, Influenza A/H3, Influenza A/H1-2009, Influenza B, Legionella pneumophila, Mycoplasma pneumoniae, Parainfluenza virus 1, Parainfluenza 2 virus, Parainfluenza 3 virus, Parainfluenza 4 virus, Pseudomonas aeruginosa, Rhinovirus A, Rhinovirus B, Rhinovirus C, Staphylococcus coagulase negative epidermis, Staphylococcus coagulase negative haemolyticus, Staphylococcus coagulase negative lugdunensis, Staphylococcus agalactiae (Group B Strep), Staphylococcus pyogenes (Group A strep), ACT, MIR, FOX, ACC, dfr A1, dfr A5, sul 1 probe, sul 2 probe, IMP, NDM, VIM Groups, OXA-48, OXA-51, qnrA 1, qnrA2, qnrB2, tet B and tet M viruses and bacteria in human in thereof consisting essentially of administering effective amounts of RAAS Nutritionals food functional dietary supplements derived from soybean (Glycine max) grape (Vitis vinifera) and rice (Oryza sativa)

2. A method of prophylaxis and therapeutic treatment of SARS-CoV-2, Acinetobacter baumanii, Bordetella pertussis, parapertusis, bronchiseptica, Chlamydia trachomatis, Coronavirus-299E, Coronavirus NL63, Coronavirus OC43, Coronavirus HKU1, Cytomegalovirus CMV, Cytomegalovirus Human Herpesvirus-5, Enterobacter aerogenes, Enterobacter cloacae, Enterovirus D, Fusobacterium nucleatum, Fusobacterium necrophorum, Herpes simplex virus 1 (HSV-1), Herpes simplex virus 2 (HSV-2), Human metapneumovirus, Klebisella pneumoniae, Klebisella oxytoca, Moraxella catarrhalis, Meisseria gonorrhoeae, Proteus mirabilis, Proteus vulgaris, Respiratory syncytial virus, Serratia marcescens, Staphylococcus aureus, Streptococcus pneumoniae, Treponema pallidum, CTX-M1 (15), CTX-M2 (2), CTX-M9 (9), CTX-M8/25 groups, ermB, ermC, mefA, mecA, Per-1NEB-1/GES-1 Groups, SHV, KPC Groups, VanA, VanB, Adenovirus Respiratory HAdV-B, Candida Albicans, Candida glabrata, Candida parapsilosis, Candida tropicalis, Chlamydophila pneumoniae, EBV(Epstein Barr Virus/Human Herpes Virus 4), Enterovirus A, Enterovirus B, Enterovirus C, Escherichia coli, Haemophilus influenzae, Human herpesvirus 3 (varicella zoster virus), Influenza A, Inluenza A/H1, Influenza A/H3, Influenza A/H1-2009, Influenza B, Legionella pneumophila, Mycoplasma pneumoniae, Parainfluenza virus 1, Parainfluenza 2 virus, Parainfluenza 3 virus, Parainfluenza 4 virus, Pseudomonas aeruginosa, Rhinovirus A, Rhinovirus B, Rhinovirus C, Staphylococcus coagulase negative epidermis, Staphylococcus coagulase negative haemolyticus, Staphylococcus coagulase negative lugdunensis, Staphylococcus agalactiae (Group B Strep), Staphylococcus pyogenes (Group A strep), ACT, MIR, FOX, ACC, dfr A1, dfr A5, sul 1 probe, sul 2 probe, IMP, NDM, VIM Groups, OXA-48, OXA-51, qnrA1, qnrA2, qnrB2, tet B and tet M viruses and bacteria in human in thereof comprising administering to the patient an efficacious dossage of the RAAS Nutritionals, LLC food functional dietary supplement as claimed in claim 1.
